(19) United States
(12) Patent Application Publication
(10) Pub. No.: US 2024/0315604 A1
(43) Pub. Date: Sep. 26, 2024

(54) HEARING LOSS MONITORING SYSTEM USING AUGMENTED REALITY SMART GLASSES

(71) Applicant: Meta Platforms Technologies, LLC, Menlo Park, CA (US)

(72) Inventor: Anjali Induchoodan Menon, Redmond, WA (US)

(21) Appl. No.: 18/399,965

(22) Filed: Dec. 29, 2023

Related U.S. Application Data
(60) Provisional application No. 63/492,202, filed on Mar. 24, 2023.

(51) Int. Cl. A61B 5/12; A61B 5/00

(52) U.S. Cl. CPC A61B 5/125; A61B 5/6803; A61B 5/7275; A61B 2560/0242; H04R 1/1083; H04R 2499/15

(57) ABSTRACT

A wearable device for monitoring the hearing of a user of the wearable device is provided. In one aspect, the wearable device may include a sensor and a processor. The processor may be configured to receive sensor data from the sensor. The sensor data may include a primary noise and an ambient noise in the environment of the user. The processor may be configured to determine a baseline listening-effort-to-ambient-noise ratio (LEANR) based on the primary noise and the ambient noise in the environment. The processor may be configured to determine multiple additional LEANRs. The processor may be configured to determine, over a predetermined time interval, an LEANR trend for the baseline LEANR and the multiple additional LEANRs. The processor may be configured to determine, based on the LEANR trend, a loss of hearing of the wearable device user. A method and a non-transitory machine-readable storage medium are also disclosed.

### CROSS-REFERENCE TO RELATED APPLICATIONS

The present application claims the benefit of priority under 35 U.S.C. § 119 from U.S. Provisional Patent Application Ser. No. 63/492,202 entitled “HEARING LOSS MONITORING SYSTEM USING AUGMENTED REALITY SMART GLASSES,” filed on Mar. 24, 2023, the disclosure of which is hereby incorporated by reference in its entirety for all purposes.

### BACKGROUND

### Technical Field

The present disclosure generally relates to health tracking. More particularly, the present disclosure relates to the use of wearable technology to detect hearing loss.

### Related Art

The decibel (dB) is a measure of how loud a sound is. The typical hearing range for an adult is from −10 to 15 dB. A person may be considered to have hearing loss—that is, a partial or total inability to hear—if that person is unable to hear at a threshold of 16 dB or better (i.e., lower).

Hearing loss severity may be categorized according to seven (7) ranges. Individuals with slight hearing loss cannot hear sounds below 16 to 25 dB; mild, 26 to 40 dB; moderate, 41 to 55 dB; moderately severe, 56 to 70 dB; severe, 71 to 90 dB; and profound, 91 or greater dB. For context, the sound level of a whisper is around 30 dB; a normal conversation, 60 dB; a shout, 90 dB; and thunder or an ambulance siren, 120 dB. So-called “disabling” hearing loss refers to hearing loss greater than 35 dB. Worldwide, one (1) out of five (5) people live with hearing loss. Hearing loss may occur at any age, may occur in one or both ears, and may be temporary or permanent. Causes may include infection, injury, genetic variations, allergies, wax buildup, aging, or exposure to loud noises (i.e., over 70 dB for prolonged periods or over 120 dB for brief periods).

Although hearing loss may occur rapidly, that is, all at once or over a few days, it is common for hearing to worsen gradually, over months or years. When hearing loss occurs gradually, an individual is less likely to notice and to seek intervention. Additionally, such gradual hearing loss may adversely affect an individual's ability to safely or to satisfactorily engage with an augmented reality (AR) environment.

### SUMMARY

The subject disclosure provides for a wearable device that may detect hearing loss in a user of the wearable device. The disclosure concerns the problem of monitoring hearing over time and identifying changes that suggest hearing loss. The disclosed solution addresses the problem by leveraging the sensors of a set of augmented reality (AR) smart glasses to determine the effort a user puts toward hearing a particular sound and to determine whether the person tries harder than previously or harder than most others to hear the particular sound.

According to certain aspects of the present disclosure, a wearable device for monitoring the hearing of a user of the wearable device is provided. The wearable device may include multiple sensors and multiple processors. The processors may receive sensor data from the sensors. The sensor data may include a primary noise and an ambient noise in the environment of the user. The processors may determine a baseline listening-effort-to-ambient-noise ratio (LEANR) based on the primary noise and the ambient noise in the environment. The processors may determine additional LEANRs. The processors may determine, over a predetermined time interval, an LEANR trend for the baseline LEANR and the additional LEANRs. The processors may determine, based on the LEANR trend, a loss of hearing of the user of the wearable device.

According to other aspects of the present disclosure, a method for monitoring the hearing of a user of a wearable device is provided. The method may include receiving, by multiple processors, sensor data from multiple sensors. The sensor data may include a primary noise and an ambient noise in the environment of the user. The method may include determining a baseline listening-effort-to-ambient-noise ratio (LEANR) based on the primary noise and the ambient noise in the environment. The method may include determining a plurality of additional LEANRs. The method may include determining, over a predetermined time interval, an LEANR trend for the baseline LEANR and the plurality of additional LEANRs. The method may include determining, based on the LEANR trend, a loss of hearing of the user of the wearable device.

According to yet other aspects of the present disclosure, a non-transitory machine-readable storage medium is provided. The non-transitory machine-readable storage medium may include instructions that, when executed by multiple processors of a wearable device, cause the wearable device to perform operations. The operations may include receiving, by multiple processors, sensor data from multiple sensors. The sensor data may include a primary noise and an ambient noise in the environment of the user. The operations may include determining a baseline listening-effort-to-ambient-noise ratio (LEANR) based on the primary noise and the ambient noise in the environment. The operations may include determining a plurality of additional LEANRs. The operations may include determining, over a predetermined time interval, an LEANR trend for the baseline LEANR and the plurality of additional LEANRs. The operations may include determining, based on the LEANR trend, a loss of hearing of the user of the wearable device.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates a perspective view of a set of augmented reality (AR) smart glasses for monitoring the hearing of a user, according to certain aspects of the disclosure;

FIG. 2 illustrates a network architecture including the AR smart glasses shown in FIG. 1 and illustrates a block diagram of the AR smart glasses shown in FIG. 1; and

FIG. 3 illustrates a process flow diagram of a method that may be performed by the AR smart glasses shown in FIGS. 1 and 2.

### DETAILED DESCRIPTION

Hearing loss may occur rapidly—over days, hours, or even minutes—but hearing loss may also occur gradually, over weeks, months, or years. When hearing loss occurs gradually, an individual may be less likely to take note and to seek help that could potentially arrest or even reverse the progression. Unfortunately, an individual may lack the training or the tools to regularly and accurately assess their hearing themselves, or they may lack the means to seek professional assessment at regular enough intervals. Moreover, an individual may find prohibitive the amount of time required to monitor their hearing themselves or to have it monitored by others. A solution to this problem is a wearable device that may be kept on the individual's person and may make use of the device's sensors to monitor the individual's hearing. Such a solution is embodied herein by a set of augmented reality (AR) smart glasses for monitoring the hearing of a user.

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regards to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the included clauses. Various aspects of the subject technology will now be disclosed according to particular but non-limiting examples. Various embodiments described in the present disclosure may be carried out in different ways and variations, and in accordance with a desired application or implementation.

In the following detailed description, numerous specific details are set forth to provide a full understanding of the present disclosure. It will be apparent, however, to one ordinarily skilled in the art, that embodiments of the present disclosure may be practiced without some of the specific details. In other instances, well-known structures and techniques have not been shown in detail so as not to obscure the disclosure.

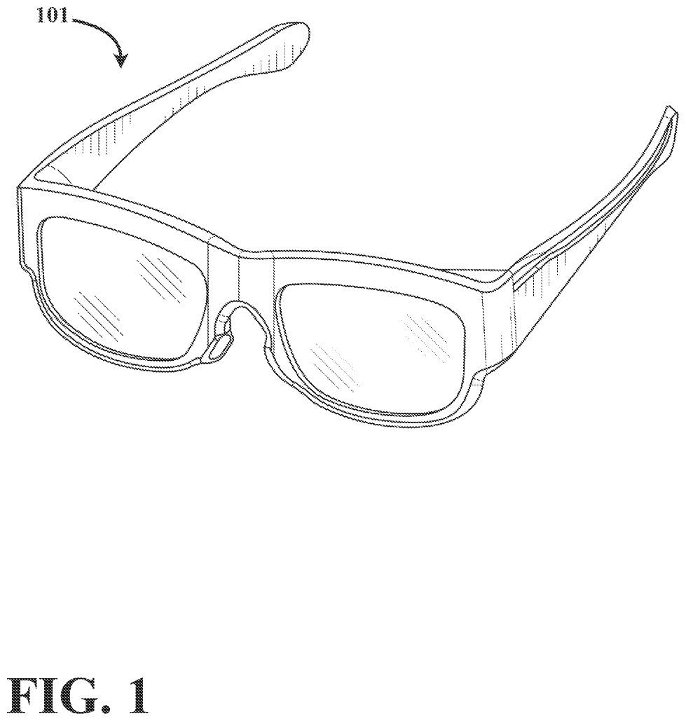

Turning now to the figures, FIG. 1 illustrates a perspective view of a set of augmented reality (AR) smart glasses 101 for monitoring the hearing of a user. The AR smart glasses 101 may include cinema display glasses, which may be configured to display a virtual screen, or extended reality (XR) glasses, which may be configured to overlay two- or three-dimensional (2-D or 3-D) digital content onto real-world objects, or a combination of the two. The AR smart glasses 101 may include a wireless connectivity module to implement wireless networks (e.g., Wi-Fi, Bluetooth®, or cellular). The AR smart glasses 101 may be used for other applications, e.g., security, entertainment, education, or training. The AR smart glasses 101 may include other aspects, e.g., battery, touchpad, temples, or headband.

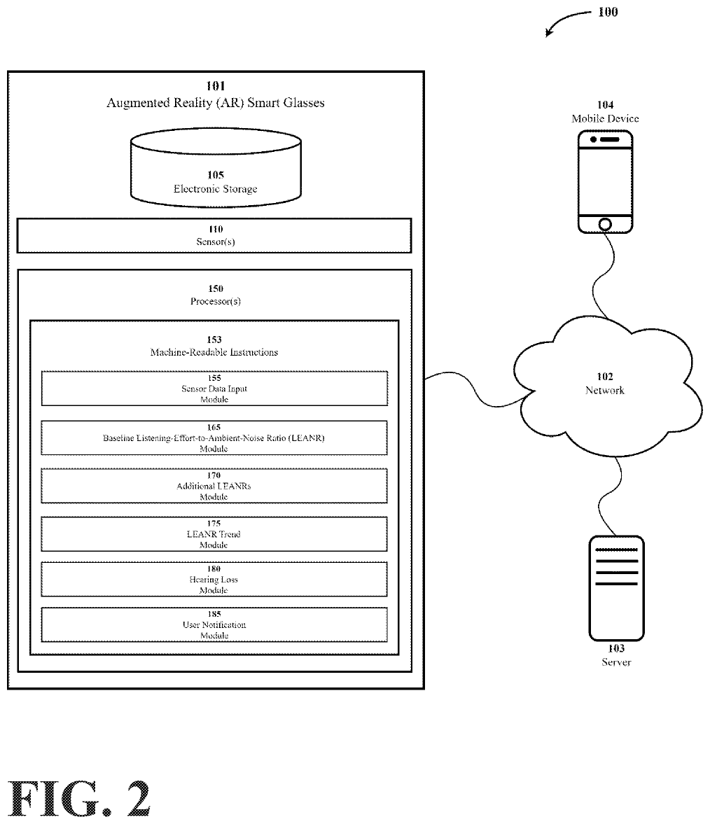

FIG. 2 illustrates a network architecture 100 including the AR smart glasses 101 shown in FIG. 1 and illustrates a block diagram of the AR smart glasses 101 shown in FIG. 1. The network 102 interconnecting the elements of the network architecture 100 may correspond to a communication network. The communication network may be any one of a local area network (LAN), wide area network (WAN), the Internet, a direct peer-to-peer (P2P) network (e.g., Bluetooth®), indirect P2P network, and the like. The server 103 may correspond to one or more server computers. The server 103 may be located locally or remotely. The server 103 may host applications installed on the mobile device 104 or the AR smart glasses 101. An application may allow the user to receive a notification of the status of their hearing. The mobile device 104 may include one or more mobile devices, which may include laptop computers, E-readers, tablets, handheld video gaming consoles, smart watches, and smartphones. The mobile device 104 may host one or more applications that notify a user of the AR smart glasses 101 of the status of their hearing.

The AR smart glasses 101 include electronic storage 105, sensor(s) 110, and processor(s) 150. The processor(s) 150 includes machine-readable instructions 153 that the processor(s) 150 may execute to operate the following modules: a sensor data input module 155; a baseline listening-effort-to-ambient-noise ratio (LEANR) module 165; an additional LEANRs module 170; an LEANR trend module 175; a hearing loss module 180; and a user notification module 185. As used herein, the term “module” may refer to any component or set of components that perform the functionality attributed to the module. This may include one or more physical processors during execution of processor readable instructions, the processor readable instructions, circuitry, hardware, storage media, or any other components.

The electronic storage 105 may include non-transitory storage media that electronically stores data. The electronic storage media of electronic storage 105 may include one or both of system storage that is substantially non-removable from the AR smart glasses 101 and system storage that is removable from the AR smart glasses 101. The removable storage may be connectable to the AR smart glasses 101 via a port (e.g., USB port, SD card slot, and the like) or a drive (e.g., a disk drive).

The sensor(s) 110 may include one or more sensors. The sensor(s) 110 may include the following types: structured-light sensors, time of flight (ToF) cameras, thermal sensors, ambient light sensors, video cameras, inertial measurement units (IMUs), directional microphones, vertical-cavity surface-emitting laser (VCSEL) based light detection and ranging (LiDAR) sensing, and binocular depth sensing.

The processor(s) 150 may include one or more processors, which may be configured to provide data processing capabilities in the AR smart glasses 101. The processors(s) 150 may include one or more of a digital processor, an analog processor, a digital circuit designed to process data, an analog circuit designed to process data, a state machine, or other mechanisms for electronically processing data. The processor(s) 150 may be configured to execute one or more of modules 155, 165, 170, 175, 180, 185, or other modules.

The sensor data input module 155 may detect the status of input signals and receive sensor data from the sensor(s) 110, wherein the sensor data may include a primary noise and an ambient noise in the environment of the user of the AR smart glasses 101. A primary noise may be a sound on which the user of the AR smart glasses 101 intends to focus. An ambient noise may be all sound other than the primary noise. According to some aspects, the sensor data may further include the following: eye gaze of the user of the AR smart glasses 101, head pose of the user of the AR smart glasses 101, and images of the environment of the user of the AR smart glasses 101.

The baseline listening-effort-to-ambient-noise ratio (LEANR) module 165 may determine a baseline listening-effort-to-ambient-noise ratio (LEANR) based on the primary noise and the ambient noise in the environment. A primary noise and an ambient noise may possess the same or different degrees of loudness, and the primary noise may possess a larger or smaller degree of loudness than the ambient noise. According to some aspects, the LEANR may quantify listening difficulty at given ambient noise conditions by considering sensor data such as eye gaze, head pose, or environment images. For example, a user may acknowledge someone calling their name at a given speaking volume and in the presence of a given ambient noise level by looking toward that person. If the user begins to take longer to look toward a person calling their name at that given speaking volume and in the presence of that given ambient noise level, then the longer time could suggest hearing loss. According to some aspects, a baseline LEANR may be determined based on comparison between the primary noise and the ambient noise in the environment of the user of the AR smart glasses 101. According to some aspects, the baseline LEANR may be determined based on a subset of additional LEANRs. According to some aspects, the baseline LEANR may be categorized based on demographic data associated with the user of the AR smart glasses 101.

The additional LEANRs module 170 may determine a plurality of additional LEANRs. An additional LEANR may be included or discarded as determined by the additional LEANRs module 170. According to some aspects, each additional LEANR may be acquired at a different time instance. The LEANR trend module 175 may determine, over a predetermined time interval, an LEANR trend for the baseline LEANR and the plurality of additional LEANRs. The trend may include one or more trends. The one or more trends may be plotted or otherwise displayed for the user in a companion application for the AR smart glasses 101. The companion application may be local or external (e.g., based in the mobile device 104) to the AR smart glasses 101. According to some aspects, the predetermined time interval may be statically defined, that is, defined over a fixed interval (e.g., month to month). According to some aspects, the time interval may be dynamically defined, that is, defined over a changeable interval (e.g., four months, followed by two weeks, followed by one year).

The hearing loss module 180 may determine, based on the LEANR trend, a loss of hearing of the user of the AR smart glasses 101. According to some aspects, the user notification module 185 may output a hearing status notification to a companion application of the AR smart glasses 101. The notification may comprise a visual indicator, such as a change in color of an LED sensor, or a vibration associated with a tactile sensor.

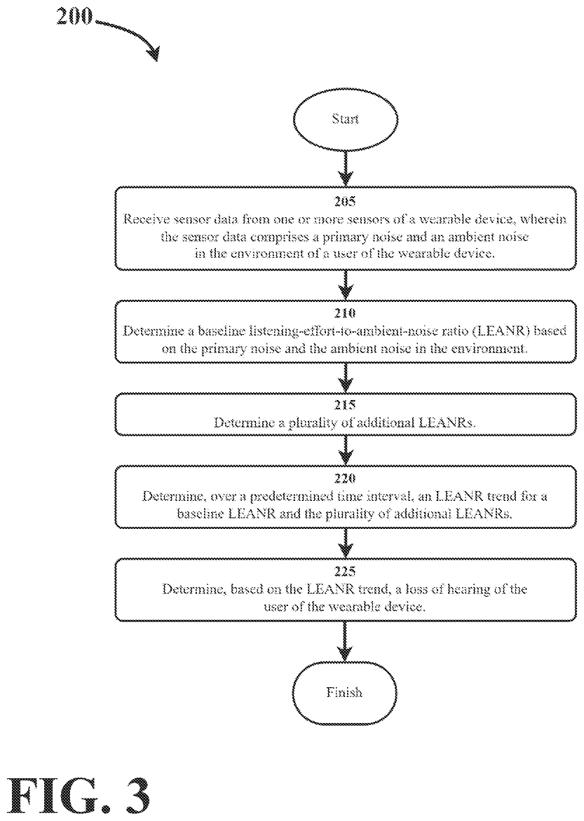

FIG. 3 illustrates a process flow diagram of a method 200 that may be performed by the AR smart glasses 101 shown in FIGS. 1 and 2. The method may begin with Block 205 and may include receiving, by one or more processors (e.g., 150), sensor data from one or more sensors (e.g., 110) of a set of AR smart glasses (e.g., 101). The sensor data may include a primary noise and an ambient noise in the environment of the user of the AR smart glasses (e.g., 101). Block 210 may include determining a baseline listening-effort-to-ambient-noise ratio (LEANR) based on the primary noise and the ambient noise in the environment. Block 215 may include determining a plurality of additional LEANRs. Block 220 may include determining, over a predetermined time interval, an LEANR trend for the baseline LEANR and the plurality of additional LEANRs. Block 225 may include determining, based on the LEANR trend, a loss of hearing of the user of the AR smart glasses (e.g., 101). According to some aspects, the method may further include outputting to a companion application of the AR smart glasses (e.g., 101) a notification of the loss of hearing of the user of the AR smart glasses (e.g., 101).

Many of the above-described features and applications may be implemented as software processes that are specified as a set of instructions recorded on a computer-readable storage medium (alternatively referred to as computer-readable media, machine-readable media, or machine-readable storage media). When these instructions are executed by one or more processing unit(s) (e.g., one or more processors, cores of processors, or other processing units), they cause the processing unit(s) to perform the actions indicated in the instructions. Examples of computer-readable media include, but are not limited to, RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In one or more embodiments, the computer-readable media does not include carrier waves and electronic signals passing wirelessly or over wired connections, or any other ephemeral signals. For example, the computer-readable media may be entirely restricted to tangible, physical objects that store information in a form that is readable by a computer. In one or more embodiments, the computer-readable media is non-transitory computer-readable media, computer-readable storage media, or non-transitory computer-readable storage media.

In one or more embodiments, a computer program product (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

While the above discussion primarily refers to microprocessor or multi-core processors that execute software, one or more embodiments are performed by one or more integrated circuits, such as application specific integrated circuits (ASICs) or field programmable gate arrays (FPGAs). In one or more embodiments, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way), all without departing from the scope of the subject technology.

It is understood that any specific order or hierarchy of blocks in the processes disclosed is an illustration of example approaches. Based upon implementation preferences, it is understood that the specific order or hierarchy of blocks in the processes may be rearranged, or that not all illustrated blocks be performed. Any of the blocks may be performed simultaneously. In one or more embodiments, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

The subject technology is illustrated, for example, according to various aspects described above. The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a configuration may refer to one or more configurations and vice versa.

In one aspect, unless otherwise stated, all measurements, values, ratings, positions, magnitudes, sizes, and other specifications that are set forth in this specification, including in the clauses that follow, are approximate, not exact. In one aspect, they are intended to have a reasonable range that is consistent with the functions to which they relate and with what is customary in the art to which they pertain. It is understood that some or all steps, operations, or processes may be performed automatically, without the intervention of a user. Method clauses may be provided to present elements of the various steps, operations, or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the included clauses. Moreover, nothing disclosed herein is intended to be dedicated to the public regardless of whether such disclosure is explicitly recited in the clauses. No clause element is to be construed under the provisions of 35 U.S.C. § 112 (f) unless the element is expressly recited using the phrase “means for” or, in the case of a method, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a clause.

The Title, Background, and Brief Description of the Drawings of the disclosure are hereby incorporated into the disclosure and are provided as illustrative examples of the disclosure, not as restrictive descriptions. It is submitted with the understanding that they will not be used to limit the scope or meaning of the clauses. In addition, in the Detailed Description, it can be seen that the description provides illustrative examples, and the various features are grouped together in various embodiments for the purpose of streamlining the disclosure. This method of disclosure is not to be interpreted as reflecting an intention that the included subject matter requires more features than are expressly recited in any clause. Rather, as the clauses reflect, inventive subject matter lies in less than all features of a single disclosed configuration or operation. The clauses are hereby incorporated into the Detailed Description, with each clause standing on its own to represent separately patentable subject matter.

The clauses are not intended to be limited to the aspects described herein but are to be accorded the full scope consistent with the language of the clauses and to encompass all legal equivalents. Notwithstanding, none of the clauses are intended to embrace subject matter that fails to satisfy the requirement of 35 U.S.C. § 101, 102, or 103, nor should they be interpreted in such a way.

What is claimed is:

1. A wearable device for monitoring the hearing of a user of the wearable device, the wearable device comprising:
one or more sensors; and
one or more processors, wherein the one or more processors are configured to:
receive sensor data from the one or more sensors, wherein the sensor data comprises a primary noise and an ambient noise in an environment of the user,
determine a baseline listening-effort-to-ambient-noise ratio (LEANR) based on the primary noise and the ambient noise in the environment,
determine a plurality of additional LEANRs,
determine, over a predetermined time interval, an LEANR trend for the baseline LEANR and the plurality of additional LEANRs, and
determine, based on the LEANR trend, a loss of hearing of the user of the wearable device.

2. The wearable device of claim 1, wherein the wearable device comprises a set of augmented reality (AR) smart glasses.

3. The wearable device of claim 1, wherein the sensor data further comprises:
a plurality of eye gazes of the user of the wearable device;
a plurality of head poses of the user of the wearable device; and
a plurality of images of the environment of the user of the wearable device.

4. The wearable device of claim 1, wherein the LEANR is determined based on comparison between the primary noise and the ambient noise in the environment of the user.

5. The wearable device of claim 1, wherein each additional LEANR is acquired at a different time instance.

6. The wearable device of claim 1, wherein the predetermined time interval is statically defined.

7. The wearable device of claim 1, wherein the predetermined time interval is dynamically defined.

8. The wearable device of claim 1, wherein the baseline LEANR is determined based on a subset of the additional LEANRs.

9. The wearable device of claim 1, wherein the baseline LEANR is categorized based on demographic data associated with the user of the wearable device.

10. The wearable device of claim 1, wherein the one or more processors are further configured to output to a companion application of the wearable device a notification of the loss of the hearing of the user of the wearable device.

11. A method for monitoring the hearing of a user of a wearable device, the method comprising:
receiving, by one or more processors, sensor data from one or more sensors, wherein the sensor data comprises a primary noise and an ambient noise in an environment of the user;
determining a baseline listening-effort-to-ambient-noise ratio (LEANR) based on the primary noise and the ambient noise in the environment;
determining a plurality of additional LEANRs;
determining, over a predetermined time interval, an LEANR trend for the baseline LEANR and the plurality of additional LEANRs; and
determining, based on the LEANR trend, a loss of hearing of the user of the wearable device.

12. The method of claim 11, wherein the wearable device comprises a set of augmented reality (AR) smart glasses.

13. The method of claim 11, wherein the sensor data further comprises:
a plurality of eye gazes of the user of the wearable device;
a plurality of head poses of the user of the wearable device; and
a plurality of images of the environment of the user of the wearable device.

14. The method of claim 11, wherein the LEANR is determined based on comparison between the primary noise and the ambient noise in the environment of the user.

15. The method of claim 11, wherein each additional LEANR is acquired at a different time instance.

16. The method of claim 11, wherein the predetermined time interval is statically defined.

17. The method of claim 11, wherein the predetermined time interval is dynamically defined.

18. The method of claim 11, wherein the baseline LEANR is determined based on a subset of the additional LEANRs.

19. The method of claim 11, wherein the baseline LEANR is categorized based on demographic data associated with the user of the wearable device.

20. The method of claim 11, wherein the one or more processors are further configured to output to a companion application of the wearable device a notification of the loss of the hearing of the user of the wearable device.

21. A non-transitory machine-readable storage medium comprising instructions that, when executed by one or more processors of a wearable device, cause the wearable device to perform operations comprising:
receiving sensor data from one or more sensors, wherein the sensor data comprises a primary noise and an ambient noise in an environment of a user;
determining a baseline listening-effort-to-ambient-noise ratio (LEANR) based on the primary noise and the ambient noise in the environment;
determining a plurality of additional LEANRs;
determining, over a predetermined time interval, an LEANR trend for the baseline LEANR and the plurality of additional LEANRs; and
determining, based on the LEANR trend, a loss of hearing of the user of the wearable device.
